(19) United States
(12) Patent Application Publication
(10) Pub. No.: US 2005/0021046 A1
(43) Pub. Date: Jan. 27, 2005

(54) Catheter tip retention device

(76) Inventor: Fertac Bilge, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(21) Appl. No.: 10/627,456

(22) Filed: Jul. 25, 2003

(51) Int. Cl. A61F 11/00

(52) U.S. Cl. 606/108

(57) ABSTRACT

A catheter tip used reduce trauma in tracking a catheter through a body lumen is attached to the end of a catheter with a ring constructed of a shape memory alloy, e.g., nitinol. The ring is shape set to a small diameter, where the ring placed around the catheter tip holds the catheter tip onto an end of the catheter that is inserted into an slot in the catheter tip.

### FIELD OF THE INVENTION

This invention relates to catheters for tracking through a body lumen and catheter tips for atraumatic tracking. In particular the invention relates to an attachment device for attaching a catheter tip to a catheter.

### BACKGROUND OF THE INVENTION

Catheters are used in a multitude of medical applications where a catheter is used to access a portion of a body through a body lumen, in order, among other things, to treat, diagnose or image a portion of a body.

Some of the uses of catheters have been in cardiovascular applications as well as many others. Most of the catheters include tapered tips so that the catheter may be tracked through a body lumen in a relatively atraumatic manner.

Many of the atraumatic tips are secured to the end of the catheter with a glue. It is difficult in manufacturing to determine the quality of the glue attachment to the catheter tip because, among other things, the curing and degree of attachment is inconsistent and unreliable. A substantial amount of testing is currently required to make sure that a catheter tip is securely fastened to the end of a catheter. A catheter tip that break off or falls off may be very dangerous if left in the body, for example, if it is free or loose within the vascular/circulatory system. Products which have two coaxially aligned tubes crimped together, similarly produce an inconsistent separation force due to the standard distribution variation in the sizes of the parts and the associated variation in clamping force achieved during the crimping process as well as the actual variation in clamping (crimping force) of plastically crimped metal band.

Accordingly, it would be desirable to provide a catheter tip that may be reliably and securely attached to a catheter.

### SUMMARY OF THE INVENTION

The present invention provides a catheter tip coupled to a catheter by a ring like mechanism manufactured of a shape memory material (superelastic alloy, e.g., nitinol).

A nitinol alloy is selected and manufactured to have a property such that the ring provides a predictable force sufficient to secure the tip to the catheter at least to a degree consistent with standards for catheter manufacturing. Thus the invention provides a relatively repeatable manufacturing technique and a more consistent product.

A superelastic alloy used to construct the retention device has a low temperature phase or martensitic phase and a high temperature phase or an austenitic phase. The retention device is heat set to a given diameter so that in the high temperature phase, the retention device's diameter is a relatively speaking smaller diameter. The alloy is preferably nitinol and may include additional elements, including, for example, but without limitation, platinum. In use, the ring is cooled to the temperature needed to achieve transformation to its martensitic phase (known as thermally induced martensite) and is then mechanically expanded to fit over the tip. The ring is placed around the tip and is warmed to above the superelastic alloy's phase transformation temperature, which is preferably lower than room temperature. When transformed the ring attempts to revert (shrink in size) to its first configuration (the smaller diameter ring) whereby the contraction of the ring exerts a radial clamping force all around the catheter tip and inner member to secure the catheter tip to an end of a catheter inserted into the catheter tip with an interference fit.

Superelastic alloy herein refers to an alloy having superelastic properties that include a martensitic phase which has low tensile strength and which is stable at relatively low temperatures below the martensite finish temperature and an austenite phase which has a relatively high tensile strength and which is stable at temperatures above the austenite finish temperature. Medical devices using superelastic metal alloys are described, for example in U.S. Pat. Nos. 4,665,906; 5,067,957; 5,190,546; and 6,306,141 to Jervis.

In a variation according to the invention thermal processing can be eliminated. An expansion force is applied to the ring to create a stress induced phase change to expand the ring and then when the force is released the ring reverts to its preset smaller diameter shape. When stress is applied to a superelastic material at a temperature at or above that which the transformation of the martensitic phase to the austenitic phase is complete at zero stress, the material deforms elastically until it reaches a particular stress level at which the alloy then undergoes a stress-induced phase transformation from the austenitic phase to the martensitic phase (stress induced martensite). A superelastic alloy ring stressed in this manner causes part of the alloy to begin to change to the martensitic phase. The martensitic alloy first yields elastically upon the application of additional stress and then can yield plastically with permanent residual deformation. If the load on the ring is removed before any plastic deformation has occurred, the martensite alloy elastically recovers and transforms back to the austenitic phase. As such the expansion of the ring is such that the ring does not permanently deform. Once the ring is released in position in its groove in the tip of the catheter, the ring expanding force is removed which reduces the stress so that the ring can return to its original unexpanded state by transformation back to the austenitic phase.

### BRIEF DESCRIPTION OF THE DRAWINGS

FIG. 1 is a front/side view of an attachment ring in accordance with the invention.

FIG. 2 is a cross sectional view of a catheter tip in accordance with the invention.

FIG. 3 is a schematic cross sectional view of an end of a catheter with a catheter tip of FIG. 2 attached with the attachment ring of FIG. 1.

### DETAILED DESCRIPTION

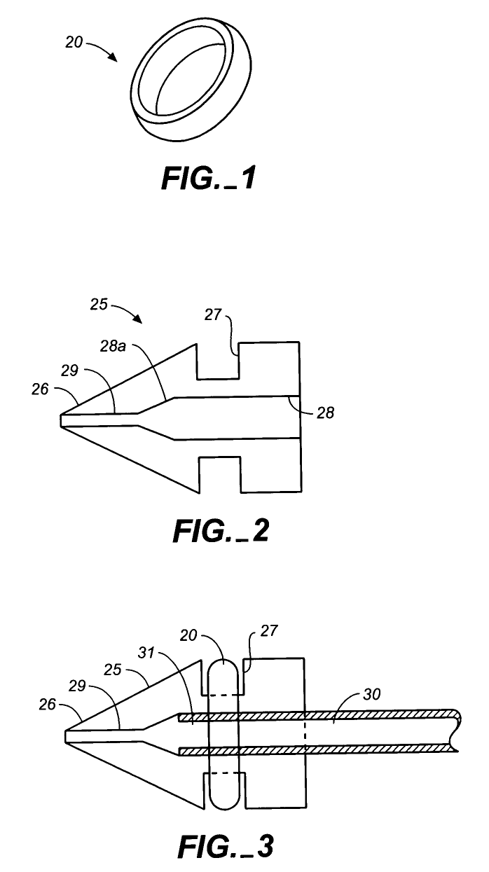

FIG. 1 illustrates a retaining ring 20. The ring 20 is constructed of a shape memory alloy such a nitinol or nitinol with other metal(s), for example, platinum, which is more radiopaque. The ring 20 has a temperature preset configuration for a first narrower diameter. The ring 20 is also shown in FIG. 3 in a first position wherein the ring 20 is shown tending to return to the first narrower diameter.

A cross sectional view of a catheter tip 25 is illustrated in FIG. 2. The tip 25 may be molded and made out of nylon, Pebax, silicone, or another soft plastic material. The tip 25 include: a tapered distal portion 26; a proximal opening 28 for receiving an end 31 of a catheter 30; a guide wire lumen 29 contiguous with the opening 28 and extending distally through the tapered distal portion 26; and a circular slot 27 formed around its circumference for receiving the retaining ring 20. The opening 28 has a tapered portion 28a which limits the axial travel of the end 31 of the catheter 30.

FIG. 3 illustrates a schematic cross section of the catheter 30 assembled with the catheter tip 25 attached to the catheter 30 (a catheter tip assembly) in accordance with the invention. The catheter tip assembly is assembled by inserting the catheter end 31 into the opening 28. In preparation for release on the catheter tip assembly, the ring 20 which has previously been heat set to a predetermined small diameter during processing is cooled to a temperature where the alloy has been transformed to a martensitic phase and is then mechanically expanded (such as by a mandrel) to a second larger diameter that permits the ring 20 to be slid over the catheter tip 25. The ring 20 is then positioned over the slot 27 and is heated (or allowed to warm up) to the temperature at which the alloy returns (transforms) to its austenitic phase. As it transforms it recovers its pre-set shape. The temperature (transformation temperature) at which the phase change occurs is preferably lower than room temperature. The ring 20 in its smaller diameter temperature set shape firmly engages and radially clamps the catheter tip 25 from within the circular slot 27. The radial force applied by of the ring 20 as it shrinks is applied to the inner member when the shape memory alloy returns to its austenite phase and tightens (or circumferentially clamps) the catheter tip 25 around the catheter end 31 to create an interference fit coupling (a tight friction fit).

One configuration for optimizing the size of the nitinol ring involves engineering it so that the force applied to the catheter tip assembly correlates to the stress and strain of the material on the plateau of the nitinol stress strain curve (as shown in the Jervis patent(s) referenced above). This means that there is a nearly constant force applied to the assembly regardless of small dimensional variations in the parts which might create large variation in stress, if non-superelastic clamping rings were used.

The tip 25 may be molded and made out of nylon, Pebax, silicone, or another soft plastic material that permit easy tracking along curves of a guidewire inside the body.

Alternatively, when thermal processing (cooling) is not used, the ring 20 when expanded may be transformed into a stress induced martensitic phase. When positioned in (or over) the circumferential slot of the tip, the expansion stress is released to allow the ring to attempt to return to its austenitic phase and as the ring shrinks and reduces its diameter it causes the tip 25 to engage the end 31 of the catheter in an interference fit type coupling.

Coupling as referred to herein means attaching or connecting together whether directly or indirectly and includes coupling two members together with or without another material or materials situated between the members such as, for example, a glue, a thin layer of another material or a material shrink wrapped onto the one or other of the members.

The shape memory alloy material is selected so that the ring of a desired dimension exerts a sufficient amount of force on the catheter tip 25 and catheter end 31 to prevent uncoupling or a break, at least according to manufacturing and product standards. Manufacturing and product standards for catheters are described in ISO 10555-1:1995(E) section 4.5, table 1 and annex B where the standard requirement for force at break at a junction, and the test for determining such force at break are described.

Although this detailed description sets forth particular embodiments according to the invention, various other catheter systems and applications are contemplated.

While the invention has been described with reference to particular embodiments, it will be understood to one skilled in the art that variations and modifications may be made in form and detail without departing from the spirit and scope of the invention.

1. A catheter tip retention device comprising:
a catheter tip having an outer circumferential wall and an opening through the outer circumferential wall for receiving an end of a catheter; and
a retaining ring constructed of a shape memory material set to a first configuration having a first diameter, wherein the retaining ring is configured to be expanded to a second configuration having a second diameter greater than the first diameter so that the ring has a circumference greater than the outer circumferential wall of the catheter tip, and so that the ring may be positioned over the outer circumferential wall of the catheter tip; and wherein the retaining ring is configured to return towards its first diameter thereby coupling the end of the catheter to the catheter tip.

2. The catheter tip retention device of claim 1 wherein the ring comprises an alloy of nickel and titanium.

3. The catheter tip retention device of claim 1 wherein the retaining ring is in an austenitic phase in the first configuration.

4. The catheter tip retention device of claim 1 wherein the retaining ring is in a temperature induced martensitic phase in the second configuration.

5. The catheter tip retention device of claim 1 wherein the retaining ring is in a stress induced martensitic phase in the second configuration.

6. The catheter tip retention device of claim 3 wherein the shape memory alloy has a phase transformation temperature of below about 68 degrees Fahrenheit.

7. The catheter tip retention device of claim 1 further comprising a catheter, wherein the catheter has an end.

8. The catheter tip retention device of claim 7 wherein the end of the catheter comprises an inner member of a catheter.

9. The catheter tip retention device of claim 7 wherein the ring causes an interference fit between the catheter tip and the end of the catheter.

10. The catheter tip retention device of claim 1 wherein the catheter tip comprises a slot in the outer circumference of the catheter tip configured to receive the ring.

11. A catheter tip retention device comprising:
a catheter tip means for tracking a catheter through a body lumen, the catheter tip means having an outer circumferential wall means and an receiving means through the outer circumferential wall means for receiving an end of a catheter means; and
a retaining means for coupling the catheter tip means to an end of a catheter means, wherein the retaining means comprises a superelastic means for causing the retaining means to return to a first set configuration having a first diameter from a second expanded configuration having second diameter greater than the first diameter, to thereby couple the catheter tip means to the end of the catheter means.

12. The catheter tip retention device of claim 11 wherein the shape memory means is temperature set to the first configuration.

13. The catheter tip retention device of claim 10 wherein the shape memory means comprises a spring means for causing the retaining means to return to the first set configuration.

14. A method of manufacturing a catheter comprising the steps of:
providing:
a catheter having an end;
a catheter tip having an outer circumferential wall and an opening through the outer circumferential wall for receiving the end of a catheter; and
a retaining ring constructed of a shape memory material set to first configuration having a first diameter;
inserting the end of the catheter into the opening of the catheter tip;
expanding the retaining ring to a second configuration having second diameter greater than the first diameter so that the ring has a circumference greater than the outer circumferential wall of the catheter tip;
positioning the ring over the outer circumferential wall of the catheter tip; and
causing the retaining ring to return towards its first diameter thereby coupling the end of the catheter to the catheter tip.

15. The method of claim 14 wherein the shape memory alloy has a phase transformation temperature;
wherein the step of expanding the retaining ring comprises: cooling the ring to a temperature at or below the phase transformation temperature; and
wherein the step of causing the retaining ring to return towards its first diameter comprises: warming the ring to a temperature at or greater than the phase transformation temperature.

16. The method of claim 14 wherein the step of expanding the ring to the second configuration comprises stressing the shape memory material to a stress induced martensitic phase; and wherein the step of causing the retaining ring to return towards its first diameter releasing the stress on the shape memory material whereby the shape memory material returns to an austenitic phase.
